(19) United States
(12) Patent Application Publication
(10) Pub. No.: US 2026/0062661 A1
(43) Pub. Date: Mar. 5, 2026

(54) INTERMITTENT PERFUSION FED-BATCH CULTURE

(71) Applicant: WUXI BIOLOGICS IRELAND LIMITED, Dundalk, Co Louth (IE)

(72) Inventors: Shaoxun XIANG, Wuxi, Jiangsu (CN); Le YU, Wuxi, Jiangsu (CN); Ziran TANG, Wuxi, Jiangsu (CN); Jun TIAN, Wuxi, Jiangsu (CN); Wesley WANG, Wuxi, Jiangsu (CN); Weichang ZHOU, Wuxi, Jiangsu (CN)

(21) Appl. No.: 19/104,178

(22) PCT Filed: Aug. 25, 2022
(86) PCT No.: PCT/CN2022/114820

§ 371 Date: Feb. 14, 2025

(51) Int. Cl. C12M 1/00; C12M 1/34; C12N 5/071; C12N 5/10

(52) U.S. Cl. CPC C12M 29/10; C12M 41/12; C12M 41/36; C12M 41/46; C12M 47/10; C12N 5/0602; C12N 5/10

(57) ABSTRACT

Provided is a method of intermittent perfusion fed-batch culture, comprising a fed-batch process including one or more intermittent perfusion phases during the middle to late stage to improve productivity and product quality.

### FIELD OF THE INVENTION

This invention pertains to the field of cell culture, and specifically a method of cell culture for improved productivity and product quality in recombinant protein production.

### BACKGROUND

In the past decade, a variety of cell culture modes have been developed in the manufacture of recombinant proteins such as monoclonal antibodies and fusion proteins. Among them, fed-batch culture is the most widely adopted approach for large-scale manufacturing owing to its operational simplicity and scalability. Perfusion culture, which represents another commonly used culture strategy with continuous medium exchange, often requires a specialized manufacturing design for a large amount of medium supply, a scalable cell retention system and corresponding downstream processing capability for continuous harvest, although its advantages in both productivity and product qualities are also obvious in comparison with fed-batch.

A fed-batch process usually lasts for around 14 days, which follows 4 distinct phases of growth curve: lag, exponential, stationary, and death. The cell death phase is a direct reflection of poor cell status usually caused by toxic metabolite accumulation during cell culture. The increased cell death may further be exacerbated by the environment resulted from the extensive release of proteases, reductases, glycosylases and sialidases into the cell culture, which can degrade proteins and alter protein glycan structures. Ultimately, cell death is associated with reduced product yield and early termination of culture process. In a fed-batch modality, the bolus feeding of nutrients may to some extent relieve or postpone the death of cells, but cannot fundamentally solve all the problems and concerns.

Media optimization has been widely used to improve cell viability and increase productivity. By feeding strategy adjustment or the supplementation of additives, cell viability and cell status may be maintained better. Unfortunately, not all the medium optimization efforts were effective or suitable for every cell line. Process parameter optimization, such as temperature shift strategies, is another approach that was proven to be effective to enhance cell performance.

Nevertheless, again, not all these modulations were widely feasible. Another way to maintain cell viability is by employing genetic engineering strategies to inhibit cell death, which might involve, for example, overexpression of negative regulators of apoptosis (BCL-2, BCL-xL, and MCL-1), knockout of positive regulators of apoptosis (BAK and BAX), and overexpression of HSP27, HSP70 or both to attenuate apoptosis (Matthew N. Henry et al., Biotechnology and Bioengineering. 2020; 117:1187-1203). However, cell line engineering is always subject to an intricate process of design, execution and validation. And in essence, none of the approaches above can fully resolve the underlying issue of deleterious by-product build-up which is the major root cause of poor cell performance at the middle to late stage of cell culture.

The popularity of perfusion process is largely attributable to its capability of extending cell culture duration by continuous removal of spent medium and replenishment of fresh medium. However, the current strategy involves expensive capital investment to establish all the required facilities, laborious medium preparation and high manufacturing cost of products. The disposal of the sizable amount of spent media is also a concern for manufacturers. Therefore, the application of a full perfusion process may not be a suitable option for all processes of recombinant production.

Thus, there exists the need for improved methods of cell culture, in particular mammalian cell culture, which allows for enhanced productivity and improved product quality in a more efficient way in a broader-spectrum of recombinant production processes.

### SUMMARY OF THE INVENTION

In general, provided herein is a novel cell culture process based on an Intermittent Perfusion Fed-Batch (IPFB) modality according to the present invention to improve productivity, product quality and upstream economics in recombinant protein production. The new culture modality is largely established on the basis of a conventional fed-batch modality by introducing one or more perfusion phases during the middle to late stage.

In one aspect, the present invention provides a method of culturing cells, comprising a fed-batch process including at least a first perfusion phase starting 0-7 days after temperature shift or 0-5 days after peak VCD (viable cell density).

Optionally and preferably in some instances, the method of the invention may further comprise one or more additional perfusion phases each independently starting 1 to 5 days, such as 2, 3 or 4 days, after the end of a previous perfusion phase.

Optionally and preferably in some instances, the method of the invention comprises a seed expansion stage comprising perfusion culture and/or enriched fed-batch culture to provide inoculation into said fed-batch process.

In some embodiments, the cells are host cells engineered to recombinantly express a product of interests and said method further comprises a step of harvesting the expressed product. The cells may be mammalian cells, such as CHO cells and the product of interests may be a polypeptide, such as a monoclonal antibody. Accordingly, also provided is a method of producing a product of interests, comprising culturing the cell expressing said product of interests according to the method of the invention and harvesting the expressed product of interests.

The IPFB culture process according to the present invention enables extended culture duration with cells better maintained in healthy status that leads to improvement in both productivity and product quality, and meanwhile, allows for reduced medium usage and more desirable cost control in comparison with conventional and typical perfusion culture. Furthermore, the IPFB modality of the invention is conveniently accommodative to whatever processes of fed-batch culture for improved productivity and product quality.

### BRIEF DESCRIPTION OF THE FIGURES

The following drawings form part of the present specification and are included to further demonstrate certain aspects of the invention. The invention may be better understood by reference to one or more of these drawings in combination with the detailed description of specific embodiments presented herein.

FIG. 1: A schematic depiction of an exemplary setup of the IPFB process according to the invention.

FIG. 2: An illustrative time line of the IPFB process according to the invention.

FIG. 3: Cell growth profiles for cultures with medium exchange(s) and a control without medium exchange in spin tubes according to an Example herein below.

FIG. 4: Cell viability profiles for cultures with medium exchange(s) and a control without medium exchange in spin tubes.

FIG. 5: Lactate profiles for cultures with medium exchange(s) and a control without medium exchange in spin tubes.

FIG. 6: Productivity profiles for cultures with medium exchange(s) and a control without medium exchange in spin tubes.

FIG. 7: Cell growth profiles for IPFB cultures and a control fed-batch culture in 3 L bioreactors according to an Example herein below.

FIG. 8: Cell viability profiles for IPFB cultures and a control fed-batch culture in 3 L bioreactors according to the Example.

FIG. 9: Lactate profiles for IPFB cultures and a control fed-batch culture in 3 L bioreactors according to the Example.

FIG. 10: Productivity profiles for IPFB cultures and a control fed-batch culture in 3 L bioreactors according to the Example.

FIG. 11: Product purity in terms of size variants for IPFB cultures and a control fed-batch culture in 3 L bioreactors according to the Example.

FIG. 12: Product purity in terms of charge variants for IPFB cultures and a control fed-batch culture in 3 L bioreactors according to the Example.

FIG. 13: Cell growth profiles for IPFB cultures and a control fed-batch culture in 3 L bioreactors according to an Example of study on intermittent perfusion rate herein below.

FIG. 14: Cell viability profiles for IPFB cultures and a control fed-batch culture in 3 L bioreactors according to the Example.

FIG. 15: Lactate profiles for IPFB cultures and a control fed-batch culture in 3 L bioreactors according to the Example.

FIG. 16: Productivity profiles for IPFB cultures and a control fed-batch culture in 3 L bioreactors according to the Example.

FIG. 17: Cell growth profiles for IPFB cultures and a control fed-batch culture in 3 L bioreactors according to an Example of study on different intermittent perfusion modalities herein below.

FIG. 18: Cell viability profiles for IPFB cultures and a control fed-batch culture in 3 L bioreactors according to the Example.

FIG. 19: Lactate profiles for IPFB cultures and a control fed-batch culture in 3 L bioreactors according to the Example.

FIG. 20: Productivity profiles for IPFB cultures and a control fed-batch culture in 3 L bioreactors according to the Example.

### DETAILED DESCRIPTION OF THE INVENTION

### Nomenclature

As used herein, singular forms preceded by “a”, “an” and “the” include plural reference unless the context clearly dictates otherwise. As well, the terms “a” (or “an”), “one or more” and “at least one” can be used interchangeably herein.

As used herein, the term “about”, “around” or “approximately”, when preceding a numerical value, refers to a range defined by approximation of 1%, 2%, 3%, 4%, 5%, 10% or more around the specified value.

In the present disclosure, one or more features in one embodiment can be combined with any one or more features in another embodiment without departing from the spirit and concept of the present invention.

In the present disclosure, all ranges, including those defined as between two specified end values, each include the specified end values, unless specified otherwise. For example, a range between 1 and 10 means a range between 1 and 10 inclusive.

In the present disclosure, when length of a time period or duration or interval is expressed in days and a time point expressed in day, it means that the time or timing is counted or identified by days, wherein the numerals does not necessarily mean exactly multiples of 24 hours.

Unless defined otherwise, all technical and scientific terms used herein have the same meanings as commonly understood by one of ordinary skills in the fields to which this invention pertains. All publications and patents specifically mentioned herein are incorporated by reference for all purposes. All references cited in this specification are to be taken as indicative of the level of the skill in the art, which should not be construed as an admission that the invention is not entitled to antedate such disclosure by virtue of prior invention.

### Embodiments of the Invention

The present invention is built on a fed-batch process by introducing one or more runs of intermittent perfusion during the middle to late stage. FIG. 1 schematically depicts an exemplary process setup according to the present invention. A culture vessel (e.g., a bioreactor) is equipped with a retention system, whereby retained cells and optionally the product in medium are returned to the culture vessel while the permeate comprising wastes and optionally the product is removed and optionally collected. Meanwhile, fresh medium is replenished by the perfusion device connected to the culture vessel to maintain the volume of culture constant.

A conventional fed-batch process leverages discontinuous bolus feeding of nutrients without any removal of the spent medium. A Fed-batch culture is usually inoculated with a predetermined cell density and then maintained via bolus feeding of nutrients. It is noted that since there is usually no exchange of spent media in the fed-batch process, the accumulation of metabolites as the cell culture proceeds tends to exert a toxic effect on either cell proliferation or target protein production. In particular, when a high cell density is used, the toxic phenomena can be exacerbated through the mechanism of cell apoptosis.

In contrast, a typical perfusion process involves continuous medium exchange with an equal flow rate for fresh medium pump-in and spent medium pump-out while retaining high numbers of viable cells. Removing spent media while keeping cells in culture can be done via cell retention means. Nevertheless, a full perfusion process is expensive. The high costs entailed in medium supply, equipments, maintenance, spent medium disposal, etc., poses a major concern, which limits its applicability.

The Intermittent Perfusion Fed-Batch (IPFB) modality according to the present invention combines advantages from both the two modalities, including the operation simplicity of fed-batch culture and the superior cell sustainability in a perfusion process. Specifically, during the middle to late stage of a fed-batch process, one or more intermittent medium exchanges are introduced by using a cell retention device. With this approach, the toxic influence can be relieved, the cell culture duration can be further extended owing to healthier cell status and production capability thereby improved. Through limited times of medium exchange using a cell retention device during the middle to late stage of fed-batch culture, both cell viability and viable cell density (VCD) can be better maintained over the full culture duration, preferably a prolonged culture and production course, which will contribute to a higher productivity and more desirable quality of product.

As used herein, the term “intermittent perfusion (IP)” refers to the one or more phases (i.e., a period of time) of perfusion that are introduced into and are thus “intermittent” relative to the otherwise full typical fed-batch process and in contrast to a full perfusion culture process. Since perfusion is largely medium exchange with retention of cells and optionally products in the bioreactor, in the present disclosure, in the context of an IPFB culture, “intermittent perfusion” and “medium exchange” can be used interchangeably.

As used herein, in the context of a fed-batch culture or an IPFB culture, the term “middle to late stage” refers to the period after temperature shift or after the peak VCD.

In particular, the present invention provides a method of culturing cells, comprising a fed-batch process including at least a first perfusion phase starting (a) 0-7 days (such as 1, 2, 3, 4, 5 and 6 days) after temperature shift or (b) 0-5 days (such as 1, 2, 3 and 4 days) after peak VCD. Accordingly, the start of the first perfusion phase is usually (not exclusively) determined according to item (a) in presence of a temperature shift, and is determined according item (b) in absence of a temperature shift.

In some embodiments, the method of invention may comprise a temperature shift, depending on the cell lines or clones. The temperature shift may be a decrease to below 37° C. such as 33° C. or lower, such as 31° C., 30° C. or lower. In some embodiments, the temperature shift is conducted when VCD climbs to at least about 50% of peak VCD, at least about 70% of peak VCD, at least about 80% of peak VCD, or at least about 90% of peak VCD, such as about 50%-95%, about 50%-75%, about 70%-90% or about 85%-95% of the peak. In some embodiments, the temperature shift is conducted at a day between day 0 and day 5 of said fed-batch process (i.e., the IPFB process), such as day 1, day 2, day 3 and day 4, based on a counting from the day of inoculation as day 0. An early temperature shift is preferred in the case of a high density inoculation. In the present disclosure, the days are counted along the fed-batch process (i.e., the IPFB process) from the day of inoculation as day 0.

As the reference for determination of time of temperature shift or the start of the first perfusion phase, the “peak VCD” may refer to a pre-determined peak VCD, that is, the peak determined in a preliminary matching experiment without temperature shift. Preliminary matching experiments are conventional to obtain informative parameters indicative of the profile of a real run. As well understood, such a pre-determined peak VCD may be identical to or very much close to the peak in real runs.

In some embodiments, the first perfusion phase starts at day 2 of said fed-batch process or later, such as day 3, day 4, day 5, day 6, day 7, day 8, day 9, day 10 or later. For example, the first perfusion phase may start at a day between day 2 and day 7, or between day 4 and day 8.

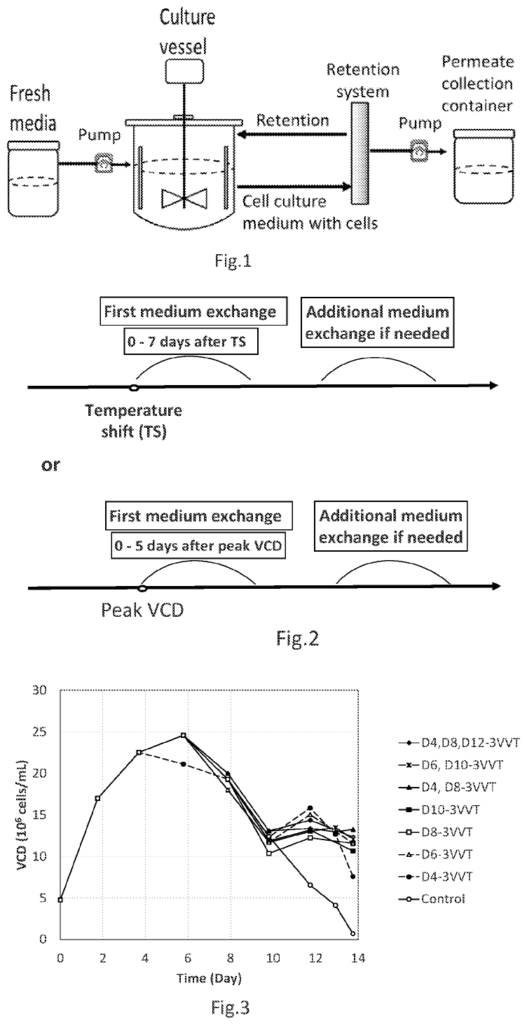

In some embodiments, the method of invention comprises more than one perfusion phases. That is, the method of the invention may optionally comprise one or more additional perfusion phases in addition to the above-said first perfusion phase for further improvement in process performance and product quality. The one or more additional perfusion phases may each independently start 1 to 5 days after the end of a previous perfusion phase, wherein the adjective “previous” means the one (n−1) immediately before the present or the subject one (n). Accordingly, for example, the method of the invention may further comprise at least a second perfusion phase starting 1 to 5 days after the end of the first perfusion phase, and optionally at least a third perfusion phase starting 1 to 5 days after the end of the second perfusion phase. In some embodiments, the one or more additional perfusion phases may each independently start 2, 3 or 4 days after the end of a previous perfusion phase. FIG. 2 depicted an illustrative timeline of the IPFB culture process according to the invention.

In some embodiments, the one or more additional perfusion phases, such as a second perfusion phase, may start at day 3 or later, such as day 5 or later, day 6 or later, day 7 or later, day 8 or later, day 9 or later, day 10 or later, day 11 or later and/or day 12 or later. In some embodiments, the one or more additional perfusion phases, such as a second perfusion phase, may start at a day between day 5 and day 12 or between day 8 and day 12 of said process. In some embodiments, the process of invention comprises a third perfusion phase, which may start at day 8 or later, such as day 9 or later, day 10 or later, day 11 or later, day 12 or later, or in some cases, a day between day 8 and day 12.

The additional runs of intermittent perfusion may enhance improvement in productivity and product quality. Notably, even for an IPFB process including multiple runs of intermittent perfusion according to the present invention, the amount of medium required for the process can be greatly reduced compared to a full typical perfusion process.

According to the present invention, IPFB applies medium exchange of up to 100% for one or multiple times during the culture period. In some embodiments, each run of medium exchange, i.e., the intermittent perfusion, is independently conducted at a perfusion rate of about 0.5-6VVT (Vessel Volume each Time) for a period of no more than 72 hours. This corresponds to an exchange ratio of 39%-100%, according to the experiential formula: exchange ratio=(1-1/e^{m})×100%, wherein “m” refers to perfusion rate in VVT. In some embodiments, each run of intermittent perfusion is independently conducted at a perfusion rate of about 1-3 VVT. In some embodiments, each run of intermittent perfusion is independently conducted for a period of about 6 to 72 hours, such as about 12-48 hours or about 24-36 hours.

The intermittent perfusion comprises replacing cell-free spent medium comprising cell waste products and toxic metabolites with fresh medium, wherein cells are retained by a cell retention device that is connected to the bioreactor via an external loop. Various cell retention techniques and corresponding devices as known useful in perfusion cultures and systems can be used. In some embodiments, the cell retention has a cut-off capable of retaining cells and the product of interests such as a target polypeptide or a monoclonal antibody in the culture vessel. For example, the cut-off may be 50 KD or less. In some embodiments, hollow fiber tangential-flow filtration, such as the standard tangential-flow filtration (TFF) or the alternating tangential-flow (ATF), is used. Hollow fiber tangential-flow filtration, especially ATF, is preferred for mammalian cells. In some embodiments, the hollow fiber tangential-flow filtration has a pore size (e.g., a nominal pore size) of 50 KD or less, which means a filter (e.g., a cartridge or column) of said pore size is used. In some embodiments, the intermittent perfusion comprises ATF as the cell retention technique and device.

In the present invention, the fed-batch process usually starts with seed inoculation at a predetermined seeding density, such as about 0.3×10⁶ to 10×10⁶ cells/mL, about 0.3×10⁶ to 50×10⁶ cells/mL, in some cases even higher. For example, the seeding density can be about 5±1.0×10⁶ cells/mL, about 10±2.0×10⁶ cells/mL, about 20±2.0×10⁶ cells/mL, about 30±2.0×10⁶ cells/mL, about 40±2.0×10⁶ cells/mL or about 50±2.0×10⁶ cells/mL. In some embodiments, a high seeding density of about 5±1.0×10⁶-10±2.0×10⁶ cells/mL is used. The high seeding density may be provided by a high seed culture density, which may be achieved by applying an intensified seed expansion method like perfusion or enriched fed-batch. Accordingly, in some embodiments, the method of the invention further comprises a seed expansion stage comprising perfusion culture and/or enriched fed-batch culture to provide inoculation into said fed-batch process.

During the culture, feed and carbon source such as glucose are supplemented periodically as designed in a typical fed-batch process. In some embodiments, feeds are added once in every two days, which may start from Day 0. In some embodiments, feed volumes are independently 2%-4% of the culture volume. In some embodiments, the glucose level is maintained between about 3-10 g/L.

Generally, the method of invention can be used for an improved harvest of biomass and/or product of interests. In some embodiments, the cultured cells are animal cells, such as mammalian cells, and in particular, CHO cells.

In some embodiments, cultured are host cells transformed to recombinantly express a product of interests and said method further comprises a step of harvesting the expressed product. In some embodiments, products of interests may be peptides, polypeptides and proteins, such as immunoglobulins and monoclonal antibodies or fragments thereof. Accordingly, also provided is a method of producing a product of interests, comprising culturing the cell expressing said product of interests according to the method of the invention and harvesting the expressed product of interests.

As used herein, the term “monoclonal”, when being used to modify an antibody, refers to the homogeneity in structure and in activity, rather than the way of generation. The monoclonal antibodies can be monovalent or multivalent (e.g., bivalent), and/or monospecific or multi-specific (e.g., bispecific). Fragments of antibody may include (Fab) fragments, F(ab′)₂ fragments, Fab′ fragments, Fv fragments, Fc fragments, variable heavy chain (VH) regions, single chain antibody fragments, including single chain variable fragments (scFv), and single domain antibodies (e.g., sdAb, sdFv, nanobody) fragments.

The present invention provides enhanced culture, especially mammalian culture, by solving certain challenges and bottlenecks common in fed-batch culture, including low culture viability, declining VCD, atypical lactate accumulation and low productivity. As demonstrated in the Examples below, the IPFB-based culture according to the present invention can reduce end lactate accumulation, improve VCD and viability sustainability, enhance productivity and provide more desirable product quality. As demonstrated in the Examples, productivity can be improved by at least 30%, desirably over 150%. From the perspective of scalability and operability in large-scale manufacturing of biologics, IPFB of the invention greatly reduces the complexity and the facility requirements for large amount of media preparation, storage and waste draining as found with perfusion culture. Additionally, the healthier cell status at harvest as rendered by IPFB is also beneficial to downstream impurity removal and potentially lessens safety concerns.

### EXAMPLES

The following Examples are provided merely for illustration, with no intends to limit scope of the invention.

### Materials and Methods

In the following Examples, a CHO-K1 cell line recombinantly expressing a monoclonal antibody of subclass IgG1 were cultured. Unless otherwise specified, the basal culture medium was Actipro medium (Hyclone, Cat No. SH31037) with 1% HT Supplement (Gibco, Cat No. 11067), and the feed medium was CB7a/CB7b (Hyclone, Cat No. SH31026 and Cat No. SH31027).

Cell density and viability were monitored via trypan blue dye exclusion method using Vi-Cell Analyzer equipped with Vi-Cell XR software version 2.04. Lactate accumulation and titer of the product mAb were monitored using CedexBio HT Analyzer.

### Example 1: Study of Effect of Medium Exchange at Different Days

To first study the effect of medium exchange(s) at different days, the experiments were as summarized in Table 1. Study No. 1 (“ST No. 1”) without medium exchange was taken as the control.

TABLE 1
Medium exchanges in spin tubes

| ST | Seeding | Feeding | Temperature | Glucose feed | Medium exchange |
|---|---|---|---|---|---|
| No. | density | strategy | shift strategy | strategy | timing |
| 1 | 4.76 × 10⁶ | D 0/2/4/6/8/10/12: | Shift to 31.0° C. when | Feed to 8 g/L | NA |
| 2 | cells/mL | 2%/4%/4%/4%/4%/ | VCD climbs to | when residue | Day 4 |
| 3 | | 4%/2% | 22.5 × 10⁶ cells/mL | glucose <6 g/L | Day 6 |
| 4 | | | (90% of peak VCD, | | Day 8 |
| 5 | | | day 4) | | Day 10 |
| 6 | | | | | Day 4, Day 8 |
| 7 | | | | | Day 6, Day 10 |
| 8 | | | | | Day 4, Day 8, Day 12 |

The cells were inoculated at the specified seeding density into a 50 ml spin tube comprising 15 ml of the basal culture medium. The feed was added at the days and the ratios according to Table 1. The medium exchanges were performed via centrifugation at 250 g for 5 minutes, followed by removing the supernatant and adding an equal volume of fresh medium in replacement. Such an exchange amounted to close to 100% medium exchange disregarding the cell pellet volume, which is equivalent to the around 3VVT perfusion rate used in further studies in bioreactors.

### Results

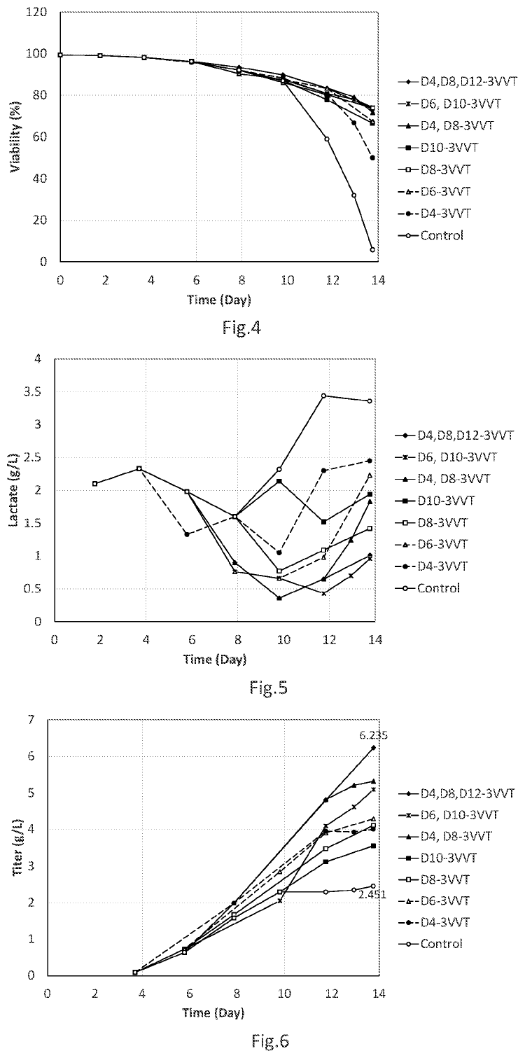

As shown in FIG. 3 and FIG. 4, though peak VCDs were largely overlapping among different processes, it is notable that processes comprising medium exchange(s) exhibited increased VCD and cell viability sustainability along the late stage, especially after Day 10. Meanwhile, medium exchange(s) also reduced residual lactate accumulation (FIG. 5). The culture in the control (i.e., ST No. 1) was terminated earlier because of low viability. In contrast, the studies with one or more medium exchanges proceed for a prolonged duration.

For the final titer of mAb product, the control ended up with a titer of 2.451 g/L, and in comparison, all the processes with one or more medium exchanges provided significantly increased titers (FIG. 6). As exhibited, single medium exchanges on Day 4, Day 6 and Day 8 increased titer to similar degrees and the single exchange on Day 10 slightly minor in comparison. Furthermore, as exhibited, increased number of exchanges further ramp up the titer of production, with ST No. 8 (“D4, D8, D12-3VVT”) giving the highest titer of 6.235 g/L. Overall, incorporation of medium exchange increased titer of product by 45% to 154%.

### Example 2: IPFB Culture in 3 L Bioreactor

For this study, an IPFB culture system was set up as schematically illustrated in FIG. 1, which included an Alternating Tangential Flow (ATF) system as the cell retention device. The experiments were as summarized in Table 2.

TABLE 2
IPFB culture in 3 L bioreactors

| Culture ID | Control | D 5 - 3 VVT | D 5, D 9 - 3 VVT |
|---|---|---|---|
| Colum | 50 KD | | |
| Medium | Non-exchange | Day 5, 3 VVT | D 5, 3 VVT and D 9, 3 VVT |
| exchange time | | | |
| Temperature | 22.8 × 10⁶ cells/ml | 22.5 × 10⁶ cells/ml | 22.3 × 10⁶ cells/ml |
| shift point¹ | (71% of peak VCD, | (68% of peak VCD, | (68% of peak VCD, |
| | day 1) | day 1) | day 1) |
| Glucose | From Day 2 to Day 13, when <6.0 g/L, raise the glucose concentration to 8.0 g/L | | |
| Feed Time | D 2/4/6/8/10/12/add 4%/4%/4%/4%/4%/2% | | |
| and ratio | | | |

| | |
|---|---|
| Seed Density | 11 × 10⁶ cells/mL |
| Initial Temp. | 36.5° C. |

Note:
¹When VCD climbs to the indicated point, shift temperature to 31.0° C.

The inoculation density into the 3 L bioreactors was 11×10⁶ cells/mL, which was realized by perfusion culture in the seed stage. For culture “D 5-3 VVT”, the intermittent perfusion was initiated on Day 5, running at a perfusion rate of 3 VVT for 24 hours; and for culture “D 5, D 9-3VVT”, in addition to the first intermittent perfusion on Day 5, a second intermittent perfusion was started on Day 9, both running at 3VVT for 24 hours.

### Results

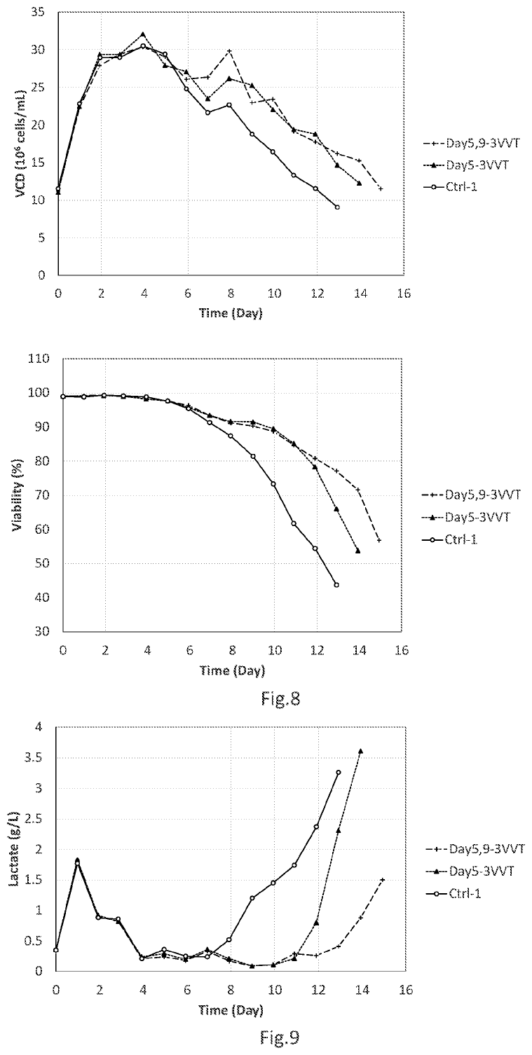

As shown in FIG. 7 and FIG. 8, though peak VCDs were largely overlapping among different processes, it is notable that processes comprising intermittent perfusion exhibited increased VCD and cell viability sustainability along the late stage. For lactate level, as shown in FIG. 9, the re-rise at a later stage started as early as Day 7 in the control; and, in contrast, the re-rise was delayed by as much as 4 days and 6 days in culture “D 5-3 VVT” comprising one run of intermittent perfusion and culture “D 5, D 9-3VVT” comprising two runs of intermittent perfusion, respectively.

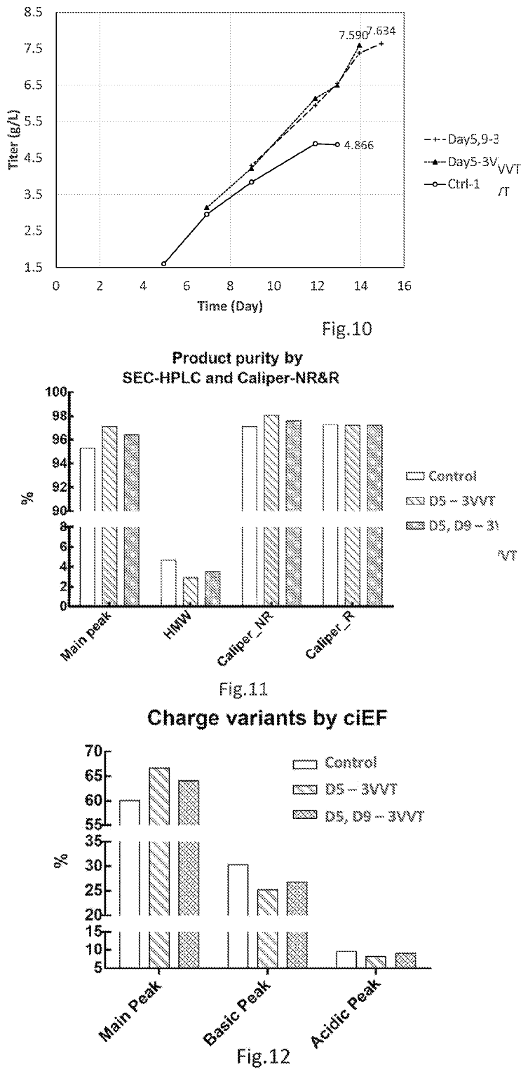

Product titers plotted over time were shown in FIG. 10. In the control, the titer increased slow, then plateaued since Day 12 and ended up with the value of 4.866 g/L; and, in contrast, in the two IPFB cultures, the titers kept increasing at a steeper slope for an extended duration till the end of culture, giving a final tier of 7.590 g/L and 7.634 g/L respectively, which represented an increase of more than 55% and an increase of more than 67% in productivity compared to the control, respectively. Further, as seen, the culture comprising two runs of intermittent perfusion provided an even higher titer than the culture comprising one.

Product quality was examined for harvest samples generated in the 3 L bioreactors after Protein A column purification. As shown in FIG. 11 and FIG. 12, purity of product in terms of both size variants and charge variants were improved in cultures with intermittent perfusion. In addition, results of N-glycan analysis were provided in Table 3. As seen, N-glycan profiles were comparable among the three tested culture modalities. These results demonstrated that IPFB can not only increase productivity but also facilitate tuning of product quality in recombinant production.

TABLE 3
N-glycan results of experiments in 3 L bioreactor

| | N-Glycan | | | | | | | | | | | | |
|---|---|---|---|---|---|---|---|---|---|---|---|---|---|
| | | | | | | G1F − GN/ | | | Man6/ | | | | |
| Culture ID | G0 − GN | G0F + GN | G0 | G0F | Man5 | G1a | G1b | G1Fa, b | 35100 | G2F | G2 | G1F + GN | Others |
| Control | 1.1 | 0.2 | 4.8 | 55.1 | 7.2 | 1.7 | 0.4 | 20.3 | 0.5 | 3.2 | 0.2 | 0.2 | 1.4 |
| D5-3VVT | 1.6 | 0.2 | 6 | 50.7 | 9 | 2 | 0.5 | 19.6 | 0.1 | 2.8 | 0.1 | 0.1 | 1.6 |
| D5, D9- | 1.7 | 0.2 | 6.3 | 50.2 | 10 | 2.1 | 0.4 | 19.4 | 0 | 2.8 | 0.1 | 0.2 | 1.6 |
| 3VVT | | | | | | | | | | | | | |

### Example 3: Effects of Intermittent Perfusion Rate

In this study, effects of perfusion rate were further studied. Two IPFB cultures in 3 L bioreactors using different perfusion rates were evaluated in comparison with a control culture. The experiments were as summarized in Table 4.

TABLE 4
Study on effects of perfusion rate in 3 L bioreactors

| Culture ID | Control | D 5 - 2 VVT | D 5 -1 VVT |
|---|---|---|---|
| Colum | 50 KD | | |
| Medium | Non-exchange | Day 5, 2VVT | Day 5, 1 VVT |
| exchange time | | | |
| Temperature | 19.7 × 10⁶ cells/ml | 19.6 × 10⁶ cells/ml | 19.4 × 10⁶ cells/ml |
| shift point¹ | (60% of peak VCD, | (60% of peak VCD, | (60% of peak VCD, |
| | day 1) | day 1) | day 1) |
| Glucose | From Day 2 to Day 13, when <6.0 g/L, raise the glucose concentration to 8.0 g/L | | |
| Feed Time | D 2/4/6/8/10/12/add 4%/4%/4%/4%/4%/2% | | |
| and ratio | | | |

| | |
|---|---|
| Seed Density | 10.3 × 10⁶ cells/mL |
| Initial Temp. | 36.5° C. |

Note:
¹When VCD climbs to the indicated point, shift temperature to 31.0° C.

### Results

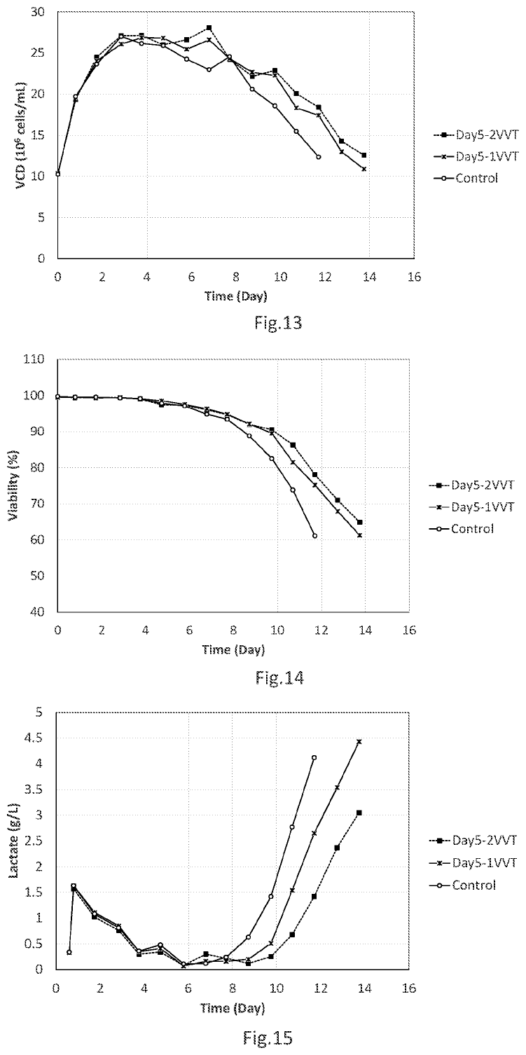

As shown in FIG. 13 and FIG. 14, IPFB cultures with intermittent perfusion at 1VVT and 2 VVT both exhibited enhanced sustainability in VCD and viability throughout the production culture compared to the control, although the peak VCDs were largely overlapping among cultures of the different modalities. The two IPFB cultures using lower perfusion rates exhibited a similar delayed lactate spike in late phase (FIG. 15), which was consistent with what was observed in the above-described examples. A lactate spike in late phase may be indicative of mitochondrial dysfunction. As reflected by the delay of lactate spikes in late phase, increased rate of medium exchange can maintain healthier cell metabolism.

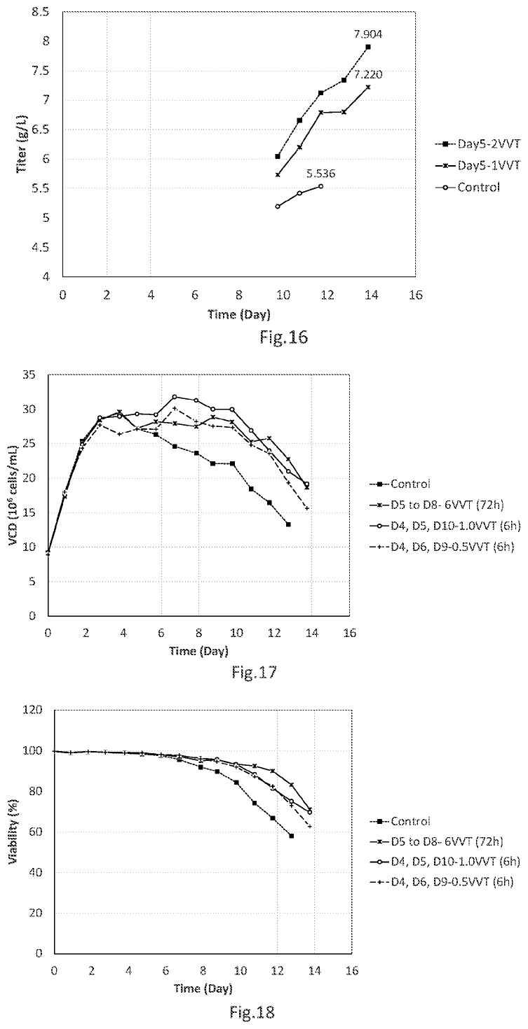

As a result of the healthy cell status, the titers of product in the IPFB cultures kept increasing rapidly till the end of culture, wherein the final protein production reached a level 30-40% higher than in the control (7.220 g/L to 7.904 g/L versus 5.536 g/L, FIG. 16). This study suggested that even with a lower perfusion rate, IPFB confers competitive advantages over the traditional fed-batch culture. Meanwhile, the process of IPFB is advantageously adaptable according to actual demand on cost control for products and to manufacturing facilities.

### Example 4: Study on Different Intermittent Perfusion Modalities

In this study, more different IP modalities, including different perfusion rates, durations and/or intervals were evaluated. The experiments were as summarized in Table 5. Therein, the culture of “Ds 5 to 8-6VVT” included one run of intermittent perfusion (IP) initiated on Day 5, running at 6VVT for 72 hours until day 8; “D4, D6, D9-0.5VVT” included 3 runs of IP at Days 4, 6 and 9 respectively, all running at 0.5 VVT for 6 hours per run, wherein the 2^{nd} and the 3^{rd} runs started 2 days and 3 days after the end of the previous run respectively; “D4, D5, D10-1.0VVT” included 3 runs of IP at Days 4, 5 and 10 respectively, all running at 1.0 VVT for 6 hours per run, wherein the 2^{nd} and the 3^{rd} runs started 1 day and 5 days after the end of the previous run respectively; and the “control” did not include IP.

TABLE 5
Different IP modalities in 3 L bioreactors

| Culture ID | Control | Ds 5 to 8 - 6VVT | D 4, D 6, D 9 - 0.5VVT | D 4, D 5, D 10 - 1.0VVT |
|---|---|---|---|---|
| Colum | 50 KD | | | |
| Temperature | 17.9 × 10⁶ cells/ml | 17.4 × 10⁶ cells/ml | 17.6 × 10⁶ cells/ml | 18.0 × 10⁶ cells/ml |
| shift point¹ | (54% of peak VCD, | (53% of peak VCD, | (53% of peak VCD, | (55% of peak VCD, |
| | day 1) | day 1) | day 1) | day 1) |
| Glucose | From Day 2 to Day 13, when <6.0 g/L, raise the glucose concentration to 8.0 g/L | | | |
| Feed Time | D 2/4/6/8/10/12/add 4%/4%/4%/4%14%/2% | | | |
| and ratio | | | | |

| | |
|---|---|
| Seed Density | 9.2 × 10⁶ cells/mL |
| Initial Temp. | 36.5° C. |

Note:
¹When VCD climbs to the indicated point, shift temperature to 31.0° C.

### Results

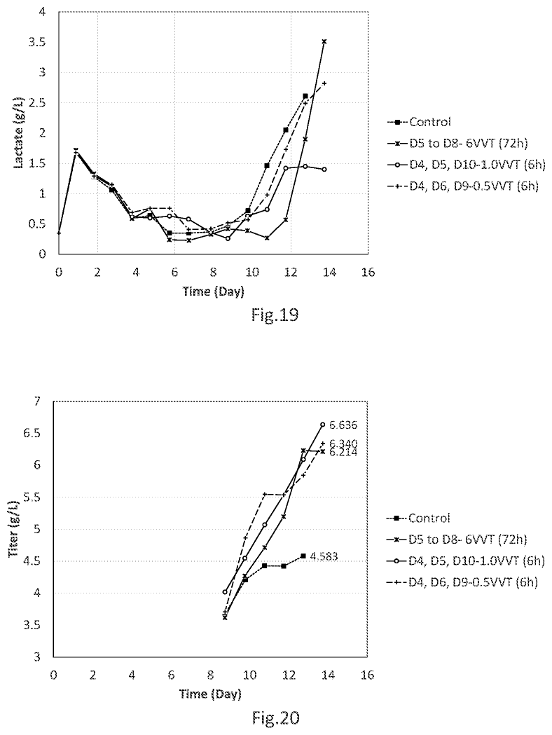

As shown in FIG. 17 and FIG. 18, all experimental groups exhibited enhanced sustainability in VCD and viability throughout the production culture compared to the control. According to FIG. 19, all experimental groups exhibited delayed lactate spike in late phase, which was consistent with what was observed in the above-described examples.

As a result of the healthy cell status, the titers of product in the IPFB cultures kept increasing rapidly till the end of culture, which provided a final titter of 6.214 g/L, 6.340 g/L and 6.636 g/L respectively, and an increased protein production level 36-45% higher than the control (4.583 g/L) (FIG. 20). This study suggests that the IPFB culture according to the present invention is flexible to expanded modulations according to actual demands, while significantly benefiting cell growth performance and productivity.

1. A method of culturing cells, comprising a fed-batch process and one or more intermittent perfusion phase, wherein a temperature shift is conducted before the first perfusion phase to decrease the temperature, the first perfusion phase starts 0-7 days after the temperature shift, and
wherein the temperature shift is conducted when viable cell density (VCD) climbs to at least 50% of peak VCD.

2. (canceled)

3. The method of claim 1, wherein said temperature shift is conducted at a day between day 0 and day 5 of said fed-batch process.

4. The method of claim 1, wherein said first perfusion phase starts at day 2 of said fed-batch process or later.

5. The method of claim 1, further comprising one or more additional perfusion phases each independently starting 1 to 5 days after the end of a previous perfusion phase.

6. The method of claim 5, wherein said one or more additional perfusion phases start at day 5 of said fed-batch process or later.

7. The method of claim 1, wherein said perfusion comprises a retention device.

8. The method of claim 1, wherein said first and said one or more additional perfusion phases run independently at a perfusion rate of 0.5-6 VVT.

9. The method of claim 1, wherein said first and said one or more additional perfusion phases run independently for a period of 6 to 72 hours.

10. The method of claim 1, wherein said method further comprises a seed expansion stage comprising perfusion culture and/or enriched fed-batch culture to provide inoculation into said fed-batch process.

11. The method of claim 1, wherein said fed-batch process start with an inoculation at a seed density of 0.3×10⁶ to 50×10⁶ cells/ml.

12. The method of claim 1, wherein said cells are mammalian cells.

13. The method of claim 1, wherein said cells are host cells transformed to recombinantly express a product of interest and said method further comprises a step of harvesting the expressed product.

14. The method of claim 13, wherein said product of interests is a polypeptide.

15. A method of producing a product of interest, comprising culturing cells expressing said product of interests according to the method of claim 1 and harvesting the expressed product of interests.

16. The method of claim 4, wherein said first perfusion phase starts at a day between day 2 and day 7 of said fed-batch process.

17. The method of claim 4, wherein said one or more additional perfusion phases start at a day between day 5 and day 12 of said fed-batch process.

18. The method of claim 7, wherein said retention device is as an Alternating Tangential Flow filtration (ATF) system or a Tangential Flow Filtration (TFF) system.

19. The method of claim 7, wherein said retention device retains the cells in the culture vessel.

20. The method of claim 14, wherein said product of interest is a monoclonal antibody.
